(19) United States
(12) Patent Application Publication
(10) Pub. No.: US 2017/0326065 A1
(43) Pub. Date: Nov. 16, 2017

(54) METHODS AND COMPOSITION FOR TREATMENT OF CARDIOVASCULAR CONDITIONS

(71) Applicants: Ezra Pharma LLC, Little Rock, AR (US); Rubicon Research Private Limited, Mumbai (IN)

(72) Inventors: Joseph A. Fix, Lawrence, KS (US); Shirish A. Shah, Phoenix, AZ (US); Pratibha S. Pilgaonkar, Mumbai (IN); Anilkumar S. Gandhi, Mumbai (IN)

(73) Assignees: Ezra Pharma LLC, Little Rock, AR (US); Rubicon Research Private Limited, Mumbai (IN)

(21) Appl. No.: 15/154,618

(22) Filed: May 13, 2016

(51) Int. Cl. A61K 9/00; A61K 9/28; A61K 9/20; A61K 31/41

(52) U.S. Cl. CPC A61K 9/0065; A61K 31/41; A61K 9/2866; A61K 9/2853; A61K 9/284; A61K 9/2009; A61K 9/2054; A61K 9/2027; A61K 9/2013; A61K 9/282

(57) ABSTRACT

The present invention relates to methods for treatment of cardiovascular conditions selected from high blood pressure, heart failure, or heart attack. The present invention further relates to a method of treating cardiovascular conditions comprising orally administering once a day to a human subject in need thereof the compound valsartan in an extended release dosage form, wherein the ratio of mean plasma concentration of valsartan provided by the extended release dosage form to the mean plasma concentration of valsartan provided by an immediate release dosage form of valsartan over 8 hour to 24 hour period after administration is greater than 1 in a single dose human pharmacokinetic study.

### FIELD OF THE INVENTION

The present invention relates to methods and composition for treatment of cardiovascular conditions. Particularly the present invention relates to methods and extended release pharmaceutical compositions of valsartan for treatment of cardiovascular conditions selected from high blood pressure, heart failure, or heart attack. Further the present invention provides a method of treating cardiovascular conditions selected from high blood pressure, heart failure or heart attack comprising orally administering once a day to a human subject in need thereof the compound valsartan in an extended release dosage form, wherein said extended release dosage form provides plasma concentration of valsartan greater than that provided by an immediate release formulation of valsartan over a time period of 8 hours to 24 hours after administration in a single dose human pharmacokinetic study. Furthermore, the present invention relates to a method of treating cardiovascular conditions comprising orally administering once a day to a human subject in need thereof the compound valsartan in an extended release dosage form, wherein the ratio of mean plasma concentration of valsartan provided by the extended release dosage form to the mean plasma concentration of valsartan provided by an immediate release dosage form of valsartan over 8 hour to 24 hour period after administration is greater than 1 in a single dose human pharmacokinetic study.

### BACKGROUND OF THE INVENTION

Various cardiovascular conditions remain the most common cause of death in the United States. The influence of circadian rhythms in cardiovascular disease is well established. It is also known that heart rate and blood pressure normally peak during the morning hours and reach a nadir in the late evening, around bedtime. The incidence of myocardial infarction, stroke, sudden cardiac death, and myocardial ischemia increases during the early morning hours. Angina attacks occur in a diurnal cycle; their occurrence is common in the hours shortly after an individual begins activity or after waking. In case of cardiovascular agents such as antihypertensive agents, the optimal levels of active have to be maintained throughout the circadian rhythms so that the heart rate, heart rhythm, and blood pressure is maintained reducing the chances of stroke, sudden cardiac death, and other myocardial ischemia diseases.

Angiotensin receptor blockers (ARBs) are well known antihypertensives that work by blocking angiotensin II thereby dilating the arterial vessels and reducing the blood pressure, and making it easier for the heart to pump. Valsartan, a selective ARB, is a well-known antihypertensive agent. Valsartan is rapidly absorbed from the gastrointestinal tract after oral administration. The absolute bioavailability of valsartan is about 25% (10-35%). This relatively low bioavailability of valsartan is primarily due to its poor solubility in the acid milieu of the gastrointestinal tract. Valsartan is an acid, and therefore, has good solubility at pH >5 and low solubility in acidic conditions of the gastrointestinal (GI) milieu. Valsartan becomes ionized in small intestine and hence cannot get absorbed in ionized form. Valsartan typically gets absorbed relatively slowly with Tₘₐₓ in the range of 2-4 hours, however following Cₘₐₓ the plasma levels starts reducing reaching to very low level by about 10-12 hours considering that the half-life of valsartan is about 6 hours. The desired antihypertensive effect therefore may not be observed when it is much needed in accordance with circardian rhythm. With the marketed immediate release formulation Diovan® there is an 80% inhibition of the pressor effect of angiotensin II infusions at valsartan peak plasma concentration (Tₘₐₓ˜3 hour) and at 24 hour there is 30% reduction in the pressor effect. Also the peak plasma concentration of valsartan is reduced to approximately 65% by 12 hours. Due to such fluctuations in the plasma levels of the active, Diovan®, normally prescribed in once-a-day regimen for the treatment of hypertension, may therefore be less optimal. Rapid decline in plasma levels of the marketed immediate release formulation Diovan® results in lack of effective blood pressure control over the entire 24 hour time period and hence makes the product short acting. This creates treatment gaps and such gaps (especially in early morning hours which are more vulnerable) increase potential of precipitating adverse cardiovascular events.

In practice, management of hypertension with Diovan® monotherapy often proceeds to twice a day dosing and/or combination therapy with a second pharmacologic agent such as hydrochlorothiazide due to inadequate blood pressure control over a 24 hour time period. Hypertension monotherapy with immediate release formulations of valsartan therefore lacks effective blood pressure control over a period of 24 hours and may cause increased number of non-responders to the therapy. Treatment with Diovan® does not provide true 24 hour blood pressure control, and tends to lose efficacy towards the end of the dosing interval, which coincides with the time that patients are at their greatest risk of precipitating adverse cardiovascular events.

A need therefore exists for treating cardiovascular conditions by administering formulations providing effective plasma concentrations of valsartan over a period of 24 hours.

US Patent Publication 20100233253 discloses an extended release gastro-retentive drug delivery system of valsartan containing a release portion containing valsartan, a gastro-retentive portion for retaining the drug delivery system in the stomach and an optional secondary portion for delivering a secondary pulse of Valsartan. WO2009084040 relates to controlled release formulation of angiotensin receptor blockers (ARB) prepared by incorporating pharmaceutically effective amounts of solubilized ARB into a gastroretentive dosage form for once a day administration. These publications, however, do not disclose providing effective plasma concentrations of valsartan over the 24 hours dose interval to avoid periods of low plasma levels of the compound and low efficacy.

The present inventors have surprisingly found that administration of extended release formulation of valsartan wherein the ratio of mean plasma concentration of valsartan provided by the extended release dosage form to the mean plasma concentration of valsartan provided by an immediate release dosage form of valsartan over 8 hour to 24 hour period after administration is greater than 1 in a single dose human pharmacokinetic study provides effective 24 hour blood pressure control. Particularly the present inventors have found that administration of extended release formulation of valsartan wherein the ratio mean plasma concentration of valsartan provided by the extended release dosage form to the mean plasma concentration of valsartan provided by an immediate release dosage form over 16-24 hour period after administration is greater than 1.5 in a single dose human pharmacokinetic study provides effective 24 hour blood pressure control and effective treatment of cardiovascular conditions.

The present invention provides method of treating cardiovascular conditions comprising administering to a human subject in need thereof extended release formulation of valsartan of the present invention that provides a prolonged absorption phase and sustained plasma concentration beyond about 12 hours or about 16 hours or about 18 hours post the administration of the dosage form. The present invention further provides method of treating cardiovascular condition comprising administering extended release formulation of valsartan that provides effective 24 hour plasma concentration and thereby provide 24 hour blood pressure control and improved organ protection.

### SUMMARY OF THE INVENTION

The present invention relates to methods of treatment of cardiovascular conditions selected from high blood pressure, heart failure, or heart attack using valsartan extended release formulations. The present invention relates to a method of treating cardiovascular conditions comprising orally administering once a day to a human subject in need thereof the compound valsartan in an extended release dosage form, wherein the ratio of mean plasma concentration of valsartan provided by the extended release dosage form to the mean plasma concentration of valsartan provided by an immediate release dosage form of valsartan over 8 hour to 24 hour period after administration is greater than 1 in a single dose human pharmacokinetic study.

### BRIEF DESCRIPTION OF THE DRAWINGS

FIG. 1 is a graph showing the ratio of plasma concentrations of valsartan provided by the extended release bilayered tablet formulation described under Example 1 to the plasma concentrations provided by the marketed valsartan immediate release formulation (Diovan®) during 24 hour dosing interval.

### DETAILED DESCRIPTION OF THE INVENTION

The present invention provides methods of treating cardiovascular conditions by administering extended release composition of valsartan that exhibits an extended protection over a time period of 24 hours. In one embodiment, the present invention provides methods that provide an improved pharmacokinetic profile over the 24 hour dosing interval and particularly during the 20 hour to 24 hour time period after administration when compared to immediate release formulations of valsartan.

The present invention provides a method of treating cardiovascular conditions selected from high blood pressure, heart failure or heart attack comprising orally administering once a day to a human subject in need thereof the compound valsartan in an extended release dosage form, wherein said extended release dosage form provides plasma concentration of valsartan greater than that provided by an immediate release formulation of valsartan over a time period of 8 hours to 24 hours after administration in a single dose human pharmacokinetic study. Furthermore, the present invention relates to a method of treating cardiovascular conditions comprising orally administering once a day to a human subject in need thereof the compound valsartan in an extended release dosage form, wherein the ratio of mean plasma concentration of valsartan provided by the extended release dosage form to the mean plasma concentration of valsartan provided by an immediate release dosage form of valsartan over 8 hour to 24 hour period after administration is greater than 1 in a single dose human pharmacokinetic study.

The term “composition” or “formulation” or “dosage form” has been employed interchangeably for the purpose of the present invention and means that it is a pharmaceutical formulation which is suitable for administration to a patient. For the purpose of the present invention, the terms “controlled release” or “sustained release” or “extended release” or “modified release” or “prolonged release” have been used interchangeably and mean broadly that the active agent is released at a predetermined rate that is different or slower than immediate release of the active agent. In one embodiment, extended release means release of valsartan from the dosage form at such a rate that when a once-a-day dose of the active agent is administered plasma concentrations (levels) of the drug are maintained within the therapeutic range but below toxic levels over a period of time of about 24 hours.

The term “single dose/dosing” means that the patient has received a single dose of the drug formulation and the drug plasma concentration has not achieved steady state. The term “mean”, when preceding a pharmacokinetic value represents the arithmetic mean value of the pharmacokinetic value taken from a population of patients unless otherwise specified (e.g. geometric mean). The term “AUC” as used herein, means area under the plasma concentration time curve, as calculated by the trapezoidal rule over the complete 24-hour interval. AUC may be further defined to refer to specific time periods relative to administration of the drug. For example AUC 20-24 refers to area under the plasma concentration time curve over the period of 20 hours to 24 hours.

In a further embodiment, the extended release formulation employed in the present invention for the treatment of hypertension comprises valsartan and at least one pharmaceutically acceptable excipient.

In one embodiment, valsartan may be present in the formulation employed in the method of the present invention in crystalline, substantially crystalline, amorphous, substantially amorphous, or dissolved form and the like or any combinations thereof. The crystalline form may have different polymorphs. All different polymorphs, solvates, hydrates, salts are within the purview of this invention. Also included within the scope of the present invention are the salts, esters, amides, prodrugs, active metabolites, analogs, and the like of valsartan. In one embodiment, valsartan is employed in the formulation employed in the method of the present invention in an amount typically ranging from about 40 mg to about 640 mg. In a further embodiment, the amount of valsartan employed in the formulation employed in the method of the present invention is from about 40 mg to about 320 mg. In another embodiment, the amount of valsartan employed in the formulation employed in the method of the present invention is from about 80 mg to about 320 mg. In one embodiment, valsartan may be present in an amount from about 1% to about 80% by weight of the composition. In one embodiment, the valsartan is present in an amount from about 2% to about 70% by weight of the composition. In another embodiment, the valsartan is present in an amount from about 5% to about 50% by weight of the composition.

In another embodiment, the extended release formulation employed in the method of treatment of the present invention comprises valsartan, at least one solubilizer, and at least one pharmaceutically acceptable excipient. In a further embodiment, the extended release formulation employed in the method of the present invention comprises valsartan, at least one solubilizer, at least one swelling polymer and at least one pharmaceutically acceptable excipient. In another embodiment, the extended release formulation employed in the method of the present invention comprises substantially solubilized valsartan, and at least one pharmaceutically acceptable excipient. In a further embodiment, extended release formulation employed in the method of the present invention comprises substantially solubilized valsartan, at least one swelling polymer and at least one pharmaceutically acceptable excipient. In a further embodiment, the formulation employed in the methods of the present invention may comprise at least a portion of dissolved valsartan. In another embodiment, the formulation employed in the methods of the present invention may comprise substantially amorphous valsartan.

In one embodiment, one or more solubilizers employed in the compositions used in the methods of the present invention may be polymeric or non-polymeric in nature. In a further embodiment, one or more solubilizers include, but are not limited to, cationic, anionic, zwitterionic or amphiphilic, or nonionic surfactants and the like or any combinations thereof. The ionic surfactants include, but are not limited to, alkylammonium salts; fusidic acid salts; fatty acid derivatives of amino acids, oligopeptides, or polypeptides; glyceride derivatives of amino acids; lecithins or hydrogenated lecithins; lysolecithins or hydrogenated lysolecithins; phospholipids or derivatives thereof; lysophospholipids or derivatives thereof; carnitine fatty acid ester salts; salts of alkylsulfates; fatty acid salts; sodium docusate; acyl lactylates; mono- or di-acetylated tartaric acid esters of mono- or di-glycerides; succinylated mono- or di-glycerides; citric acid esters of mono- or diglycerides; and the like or mixtures thereof. The amphiphilic surfactants include, but are not limited to, d-alpha-tocopheryl polyethylene glycol 1000 succinate and d-a-tocopherol acid salts such as succinate, acetate, and the like or mixtures thereof. The non-ionic surfactants include, but are not limited to, fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols or sterol derivatives; polyethylene glycol alkyl ethers; sugar esters; sugar ethers; lactic acid derivatives of mono- or diglycerides; oil soluble vitamins/vitamin derivatives; PEG fatty acid esters; polyglycerized fatty acid; polyoxyethylene-polyoxypropylene block copolymers; transesterification products of a polyol with at least one member of the group consisting of glycerides, vegetable oils, hydrogenated vegetable oils, fatty acids and sterols wherein the commonly used oils are castor oil or hydrogenated castor oil, or an edible vegetable oil such as corn oil, olive oil, peanut oil, palm kernel oil, almond oil and the commonly used polyols include glycerol, propylene glycol, ethylene glycol, polyethylene glycol, sorbitol and pentaerythritol; or mixtures thereof. In another embodiment of the present invention, the one or more solubilizers that may be employed in compositions used in the methods of the present invention include polyethylene-polyoxypropylene block copolymer (Lutrol ® series BASF) and d alpha-tocopheryl polyethylene glycol 1000 succinate (Vitamin E 25 TPGS® by Eastman) or combinations thereof.

In one embodiment, in the compositions employed in the methods of the present invention valsartan and one or more solubilizers may be employed in different ratios. In one embodiment the ratio of valsartan to solubilizers may range from about 50:1 to about 1:50. In a further embodiment, the ratio of valsartan to solubilizers is from about 20:1 to about 1:20. In another embodiment, the ratio of valsartan to solubilizer is from about 10: 1 to about 1: 10. In one embodiment, valsartan is substantially solubilized using one or more solubilizers. In a further embodiment, the term “solubilized” as used herein refers to improved or increased solubility form of valsartan. In another embodiment, the term “substantially solubilized” means valsartan is partially or completely solubilized. The term “completely or partially” as used herein refers to whether the entire or part of the valsartan dose is solubilized. In a further embodiment, the term “substantially solubilized” means more than 50% of valsartan incorporated in the extended release formulation employed in the methods of the present invention is intimately dispersed in a matrix comprising at least one solubilizer. In another embodiment, at least 75% of valsartan incorporated in the extended release formulation employed in the methods of the present invention is intimately dispersed in a matrix comprising at least one solubilizer. In one embodiment, at least 85% of valsartan incorporated in the extended release formulation employed in the methods of the present invention is intimately dispersed in a matrix comprising at least one solubilizer. In another embodiment, at least 95% of valsartan incorporated in the extended release formulation employed in the methods of the present invention is intimately dispersed in a matrix comprising at least one solubilizer. In a further embodiment, the entire amount of valsartan incorporated in the extended release formulation employed in the methods of the present invention is intimately dispersed in a matrix comprising at least one solubilizer.

In a further embodiment, the processes employed for solubilization of valsartan may include but are not limited to melt granulation, solvent treatment, wet granulation, physical mixing or spray drying and the like or combinations thereof. In a further embodiment, the process employed for the solubilization of valsartan in the composition for the treatment of cardiovascular conditions is melt granulation.

In one embodiment, the swelling polymers in the dry state or in a form that has substantial capacity for water uptake may be employed in the compositions used in the methods of the present invention. Non-limiting examples of such swelling polymers employed in the present invention include, but are not limited to, polyalkylene oxides; cellulosic polymers; acrylic acid and methacrylic acid polymers, and esters thereof, maleic anhydride polymers; polymaleic acid; poly(acrylam ides); poly(olefinic alcohol)s; poly(N-vinyl lactams); polyols; polyoxyethylated saccharides; polyoxazolines; polyvinylamines; polyvinylacetates; polyimines; starch and starch-based polymers; polyurethane hydrogels; chitosan; polysaccharide gums; alginates; zein; shellac-based polymers; polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, maltodextrin, pre-gelatinized starch and polyvinyl alcohol, and the like or mixtures thereof. In a further embodiment one or more swelling polymers employed in the extended release formulations used in the method of the present invention include, but are not limited to, polyethylene oxide, hydroxypropyl cellulose, hydroxypropyl methyl cellulose, hydroxyethyl cellulose, sodium carboxymethyl cellulose, calcium carboxymethyl cellulose, methyl cellulose, polyacrylic acid, maltodextrin, pre-gelatinized starch, polyvinyl alcohol and the like or mixtures thereof. In another embodiment, the weight percent of the swelling polymer in the final compressed dosage form is about 5 to about 95 weight percent.

In another embodiment, the pharmaceutically acceptable excipients employed in the compositions employed in the methods of the present invention include but are not limited to release modifiers, swelling enhancers, acid source, gas generating agents, binders, lubricants, diluents, disintegrants, glidants, colorants, pH modifiers, pore-formers, and the like or mixtures thereof. In a further embodiment, release modifiers employed in the compositions used in the methods of the present invention include, but are not limited to, polymeric release retardants, non-polymeric release retardants or any combinations thereof. In one embodiment, release modifiers are employed in the compositions of the present invention to control the release of valsartan and/or solubilized valsartan. In another embodiment, swelling or non-swelling polymeric release modifiers may be employed in the compositions of the present invention. Polymeric release modifiers employed in the compositions used for the methods of the present invention include, but are not limited to, cellulose derivatives; cross-linked polyvinyl pyrrolidone, polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; or any combinations thereof. Cellulose derivatives include, but are not limited to, ethyl cellulose, methylcellulose, hydroxypropylmethylcellulose (HPMC), hydroxypropyl cellulose (HPC), hydroxyethyl cellulose, hydroxymethyl cellulose, hydroxypropyl ethylcellulose, carboxymethylethyl cellulose, carboxy-ethylcellulose, carboxymethyl hydroxyethylcellulose, hydroxyethylmethyl carboxymethyl cellulose, hydroxyethyl methyl cellulose, carboxymethyl cellulose (CMC), methylhydroxyethyl cellulose, methylhydroxypropyl cellulose, carboxymethyl sulfoethyl cellulose, sodium carboxymethyl cellulose, or combinations thereof. In one embodiment release modifiers of one or more viscosity values may be employed. In another embodiment, the release modifier employed is hydroxypropyl methylcellulose. Non-polymeric release modifiers employed in the compositions used in the method of the present invention include, but are not limited to, fats, oils, waxes, fatty acids, fatty acid esters, long chain monohydric alcohols and their esters or combinations thereof. The amount of release modifier in the dosage form generally varies from about 5% to about 90% by weight of the dosage form.

In one embodiment, the swelling enhancers that may be employed in the valsartan extended release composition that are used for method of treatment of cardiovascular conditions include, but are not limited to, low-substituted hydroxypropyl cellulose, microcrystalline cellulose, cross-linked sodium or calcium carboxymethyl cellulose, cellulose fiber, cross-linked polyvinyl pyrrolidone, cross-linked polyacrylic acid, cross-linked Amberlite resin, alginates, colloidal magnesium-aluminum silicate, corn starch granules, rice starch granules, potato starch granules, pregelatinised starch, sodium carboxymethyl starch and the like or combinations thereof. In another embodiment, the content of the swelling enhancer that may be employed is about 5 to about 90 weight percent of the formulation. At concentration above 5% w/w the non-limiting list of agents listed above function as swelling enhancers and help swelling polymers to swell rapidly. In a further embodiment, acidulants may be employed in the compositions. Non-limiting examples of acidulants that may be employed in the compositions include aliphatic or aromatic, saturated or unsaturated, monobasic acid (monocarboxylic acid), dibasic acid (dicarboxylic acid) or tribasic acid (tricarboxylic acid). In one embodiment of the present invention, the acidulant used in the extended release formulation is malic acid, tartaric acid, fumaric acid, maleic acid, aspartic acid or citric acid and the like or any combinations thereof. The acidulants also function as acid source when with the gas generating agent as an effervescent couple. In a further embodiment, the extended release pharmaceutical composition may comprise at least one gas generating agent. The gas generating agents also referred to as effervescent agent aid in the formation of highly porous, preferably honeycombed structure and enhances the buoyancy of the formulation. The gas generating agent employed in the composition is selected from, but not limited to, alkali and alkaline-earth metal carbonates and bicarbonates such as sodium bicarbonate, sodium glycine carbonate, potassium bicarbonate, ammonium bicarbonate, sodium bisulfite, sodium metabisulfite, sodium carbonate, potassium carbonate and the like or combinations thereof. In one embodiment, the gas generating agent is used at concentration from about 0.5 weight % to about 25 weight % of the dosage form. Non-limiting examples of suitable binders that may be employed in the compositions of the present invention include, but are not limited to, starch, pregelatinized starch, polyvinyl prrolidone (PVP), copovidone, cellulose derivatives, such as hydroxypropylmethyl cellulose (HPMC), hydroxypropyl cellulose (HPC), carboxymethyl cellulose (CMC) and their salts and the like or combinations thereof. Non-limiting examples of diluents that may be employed in the compositions include, but are not limited to, starch, dicalcium phosphate, microcrystalline cellulose, lactose monohydrate, dextrate hydrated and the like or combinations thereof. Suitable lubricants that may be employed include, but are not limited to, magnesium stearate, calcium stearate, stearic acid, talc, sodium stearyl fumarate and the like or combinations thereof. Non-limiting examples of glidants that may be employed include, but are not limited to, colloidal silica, colloidal silicon dioxide, silica gel, precipitated silica, and the like or combinations thereof. Suitable pore forming agents such as, but not limited to, dextrates, non-GMO dextrates, lactose, sodium chloride, and the like or combinations may be employed in the formulations. Suitable adsorbents that may be used in the formulations of the present invention, include but are not limited to, silicates such as aluminum magnesium metasilicate, calcium silicate, and the like; microcrystalline celluloses and the like or combinations thereof. Suitable colorants such as, but not limited to, ferric oxide (Sicovit Red 30 E172) may be employed in the compositions used in the method of treatment of the present invention. Disintegrating agents such as, but not limited to, starch, sodium starch glycolate, pregelatinised starch, crosslinked polyvinyl pyrrolidone, cross linked carboxy methylcellulose, or ion exchange resin, may be employed in the composition if required. In one embodiment, disintegrating agents as listed herein above may be employed in the compositions of the present invention to improve the dissolution.

In a further embodiment, the formulation used in the method of the present invention is in the form of an extended release formulation. In another embodiment the extended release formulation employed in the method of the present invention is in the form of a gastroretentive dosage form. For the purpose of the present invention the term “gastroretentive” or “gastric retention” or “gastroretention” or “retained in upper gastrointestinal tract” when used with respect to the dosage form of the present invention, means that the dosage form or at least a portion thereof remains in the upper gastrointestinal tract including stomach, for about 30 minutes or more. In another embodiment, the gastroretentive dosage form employed in the method of the present invention remains in the upper gastrointestinal tract including stomach, for about 30 minutes to about 12 hours. In another embodiment extended release formulation of the present invention is in the form of a gastroretentive dosage form. In a further embodiment, gastroretentive dosage forms that are retained in the upper gastrointestinal tract for a prolonged period of time after oral administration and release the active ingredient continuously at a predetermined rate or in a sustained manner are employed for delivering valsartan.

In a further embodiment, the gastroretentive dosage form may be in the form of a monolithic system, an expanding bilayered or multilayered or in-lay system for oral administration which is adapted to deliver the drug at a predetermined rate. In one embodiment, valsartan is incorporated in monolithic matrix type of extended release gastroretentive formulation. In another embodiment, valsartan is incorporated in a bilayered gastroretentive dosage form that consists of a drug layer and a gastroretentive expanding layer wherein the drug is released at a predetermined rate from the drug layer. In a further embodiment pharmaceutical controlled release gastroretentive composition in the form of an expanding bilayered system for oral administration is provided to deliver valsartan from a first layer immediately upon reaching the gastrointestinal tract, and to deliver same or different active, from a second layer, in a sustained manner over a specific time period. The second layer is also adapted to provide expanding nature for the dosage system, thereby making the dosage system have greater retention in the stomach.

In another embodiment, the extended release formulation employed in the method of the present invention in the form of a bilayered gastroretentive dosage form exhibits an effective plasma concentration over 24 hour time period after administration. In another embodiment, the extended release formulation employed in the method of treatment of the present invention exhibits effective plasma concentration in the high risk 20 to 24 hour period after administration.

In one embodiment, the formulations employed in the present invention employed for treating cardiovascular conditions selected from high blood pressure, heart attack or heart failure are based on continuous trickling of solubilized drug in the upper gastrointestinal system for longer periods of time thereby extending the absorption phase. In a further embodiment, the sustained release formulations of valsartan are based on controlled release, gastroretention and solubilization concepts surprisingly provide effective plasma concentrations during the 20-24 hour period.

In yet another illustrative embodiment according to the invention, the extended release formulation may be optionally coated. Surface coatings may be employed for aesthetic purposes or for dimensionally stabilizing the dosage form. The coating may be carried out using any conventional technique employing conventional ingredient. A surface coating can, for example, be obtained using a quick-dissolving film using conventional polymers such as, but not limited to, hydroxypropyl methyl cellulose, hydroxypropyl cellulose, carboxymethyl cellulose, ethyl cellulose, polyvinyl alcohol, polymethacrylates or the like or combinations thereof. In one embodiment, preformed coating systems such as, but not limited to, Opadry Clear 03K19229, Opadry 200 Blue 200F105000, Kollicoat® Protect, Kollicoat® Smartseal 30D, Aquarius® MG and the like or combinations thereof may be employed as coatings for the compositions used in the present invention.

Tablets employed in the present invention may vary in shape including, but not limited to, oval, triangle, almond, peanut, parallelogram, pentagonal. It is contemplated within the scope of the invention that the dosage form can be encapsulated. Tablets used in the present invention may be manufactured using conventional techniques of common tableting methods known in the art such as direct compression, dry granulation, wet granulation and extrusion melt granulation. It is also contemplated within the scope of the invention that any process known in the art suitable for making pharmaceutical compositions in general may be employed for preparation of composition used in invention.

In another embodiment, the extended release dosage form employed for the methods of the present invention may be prepared by a process comprising: preparing solubilized valsartan by solubilization using one or more solubilizers; blending said solubilized valsartan with at least one release modifier, at least one swelling polymer and at least one pharmaceutically acceptable excipient; lubricating the blend to form a lubricated blend; compressing the blend to form a monolithic tablet. Furthermore, the extended release dosage form employed for the methods of the present invention may be prepared by a process comprising: preparing solubilized valsartan by solubilization using one or more solubilizers; blending said solubilized valsartan with at least one release modifer and at least one pharmaceutically acceptable excipient, lubricating the blend to form drug layer blend; blending at least one swelling polymer, at least one pharmaceutically acceptable excipient, lubricating the blend to form a gastroretentive layer blend; and compressing the drug layer and the gastroretentive layer to form a bilayered tablet. In one embodiment, the extended compositions of the present invention are in the form of bilayered gastroretentive dosage form comprising the active layer and the gastroretentive layer.

The extended release formulations employed for the methods according to the present invention allow for extended release of valsartan. In another embodiment the valsartan released over a period of about 24 hours. Further, within the purview of the present invention, are included formulations that comprise a combination of valsartan with other drugs or active agents which may be delivered in an immediate release or modified release manner and the use of such formulations for the treatment of cardiovascular conditions.

In one embodiment, method of treating cardiovascular conditions comprises administering extended release dosage form of valsartan that exhibits a ratio of plasma concentrations of the extended release formulation to immediate release formulation of greater than 1 over a time period of 8 to 24 hours after administration in a single dose human pharmacokinetic study. In a further embodiment, method of treating cardiovascular conditions comprises administering extended release dosage form of valsartan that exhibits a ratio of plasma concentrations of the extended release formulation to immediate release formulation of greater than 1.2 over a time period of 12 to 24 hours after administration in a single dose human pharmacokinetic study. In another embodiment, method of treating cardiovascular conditions comprises administering extended release dosage form of valsartan that exhibits a ratio of plasma concentrations of the extended release formulation to immediate release formulation of greater than 1.5 over a time period of 16 to 24 hours after administration in a single dose human pharmacokinetic study. In a further embodiment, method of treating cardiovascular conditions comprises administering extended release dosage form of valsartan that exhibits a ratio of plasma concentrations of the extended release formulation to immediate release formulation of greater than 1.5 over a time period of 20 to 24 hours after administration in a single dose human pharmacokinetic study.

In one embodiment, the present invention provides method of treating cardiovascular conditions comprising orally administering once a day to a human subject in need thereof the compound valsartan in an extended release dosage form, wherein the extended release dosage form provides plasma concentration of valsartan greater than that provided by an immediate release formulation of valsartan over a time period of 8 to 24 hours after administration in a single dose human pharmacokinetic study. In another embodiment, the present invention provides method of treating cardiovascular conditions comprising orally administering once a day to a human subject in need thereof the compound valsartan in an extended release dosage form, wherein the extended release dosage form provides plasma concentration of valsartan greater than that provided by an immediate release formulation of valsartan over a time period of 12 to 24 hours after administration in a single dose human pharmacokinetic study. In one embodiment, the present invention provides method of treating cardiovascular conditions comprising orally administering once a day to a human subject in need thereof the compound valsartan in an extended release dosage form, wherein the extended release dosage form provides plasma concentration of valsartan greater than that provided by an immediate release formulation of valsartan over a time period of 16 to 24 hours after administration in a single dose human pharmacokinetic study. In one embodiment, the present invention provides method of treating cardiovascular conditions comprising orally administering once a day to a human subject in need thereof the compound valsartan in an extended release dosage form, wherein the extended release dosage form provides plasma concentration of valsartan greater than that provided by an immediate release formulation of valsartan over a time period of 20 to 24 hours after administration in a single dose human pharmacokinetic study.

Without being bound to any theory it believed that valsartan undergoes biphasic elimination with alpha phase and beta phase. Alpha phase is very rapid (T_{1/2} 1-2 hours). Slow beta phase with T_{1/2} 7-8. Alpha phase contributes to the major decrease in the plasma levels and hence practically plasma levels drop within 3 hours after administration of an immediate release formulation. Due to this the immediate release product becomes short acting and once a day dosing regimen is not supported. It is believed that the method of the present invention comprising administering extended release formulation discussed above provides continuous drug input by extending the drug absorption phase. Since beta phase is slow; absorption rate in the later hours dominates over the elimination achieving significantly higher plasma levels compared to immediate release formulation. This culminates into prolonged effect with effective plasma concentrations over the 24 hour time period and better treatment of cardiovascular conditions.

In a further embodiment, the present invention further provides a method of treating cardiovascular conditions comprising orally administering once a day to a human subject in need thereof the compound valsartan in an extended release dosage form, wherein the extended release dosage form provides at least 25% more area under the curve compared to those provided by immediate release formulation of valsartan over the period of 16 -24 hours after administration in a single dose pharmacokinetic study. In another embodiment, the present invention further provides a method of treating cardiovascular conditions comprising orally administering once a day to a human subject in need thereof valsartan in an extended release dosage form wherein the extended release dosage form provides at least 20% of the total area under the curve (AUC) in the 16-24 hour time period after administration in a single dose pharmacokinetic study. In a further embodiment, the present invention provides method of treating cardiovascular conditions comprising orally administering once a day to a human subject in need thereof valsartan in an extended release dosage form wherein the extended release dosage form provides an area under the curve that is comparable to immediate release Diovan formulation. In another embodiment, the present invention involves redistribution of area under the curve when compared with the area under the curve obtained with Diovan.

In another embodiment the formulation used in the method of the present invention may be taken with a meal, thereby enabling patients to dose in the morning with breakfast simplifying dosing in that it can be dosed in the morning with a breakfast, which tends to improve patient compliance.

In a further embodiment is provided the use of extended release pharmaceutical composition of valsartan for the method of treatment of the present invention for the manufacture of a medicament for treatment of cardiovascular conditions selected from hypertension, heart failure and heart attack. In one embodiment, the present invention provides a method for treatment of. In another embodiment, the present invention provides a method for reducing the risk of fatal and nonfatal cardiovascular events, primarily strokes and myocardial infarctions, comprising administering to the subject in need thereof extended release pharmaceutical compositions of valsartan of the present invention exhibiting a ratio of plasma concentrations of the extended release formulation to immediate release formulation of greater than 1 over a time period of 8 to 24 hours after administration in a single dose human pharmacokinetic study. In another embodiment, the methods of treatment of cardiovascular conditions of the present invention can also be employed for the treatment of other conditions such as, but not limited to, angina, myocardial infarction, diabetic cardiac myopathy, renal insufficiency, arteriosclerosis, diabetic nephropathy, peripheral vascular disease, stroke, left ventricular hypertrophy, or cognitive dysfunction, and the like.

While the present invention has been described in terms of its specific embodiments, certain modifications and equivalents will be apparent to those skilled in the art and are intended to be included within the scope of the present invention. The invention is further illustrated by the following examples, which are for illustrative purposes and should not be construed as limiting the scope of the invention in any way.

### EXAMPLES

### Example 1

This example describes extended release valsartan formulation in Table 1.

TABLE 1

| | |
|---|---|
| Ingredients | mg/unit |

Core Tablets
Active layer

| | |
|---|---|
| Valsartan | 160 |
| Vitamin E polyethylene glycol | 80 |
| succinate | |
| Poloxamer | 80 |
| Microcrystalline Cellulose | 135 |
| Hydroxypropyl methylcellulose | 110 |
| Calcium Silicate | 120 |
| Crospovidone | 35 |
| Fumaric Acid | 78 |
| Dextrates | 60 |
| Colloidal Silicon Dioxide | 10 |
| Magnesium Stearate | 20 |
| Ferric Oxide | 2 |

Gastroretentive layer

| | |
|---|---|
| Polyethylene Oxide | 119 |
| Hydroxypropyl methyl cellulose | 119 |
| Hydroxyethyl Cellulose | 59 |
| Crospovidone | 120 |
| Microcrystalline Cellulose | 29 |
| Polyvinylpyrrolidone | 33 |
| 1-vinyl-2-pyrrolidone and vinyl acetate | 13 |
| copolymer | |
| Sodium Bicarbonate | 33 |
| Anhydrous Citric Acid | 10 |
| Magnesium Stearate | 5 |
| Isopropyl Alcohol | q.s. |
| Purified Water^{#} | q.s. |
| Total | 1430 |

Coating System

| | |
|---|---|
| Polyvinyl alcohol-based Opadry 200 | 45 |
| Blue 200F105000 | |
| Purified water^{#} | q.s. |
| Excipients (imprinting material) | |
| Opacode Black S-1-17823 | 0.104 |
| Isopropyl alcohol^{#} | q.s. |
| Total | 1490 |

^{#}expelled during manufacturing process, not part of the final product

Process of Preparation: Valsartan was added to molten poloxamer and vitamin E polyethylene glycol succinate in a low shear mixer and mixed well. A part of microcrystalline cellulose, calcium silicate, fumaric acid and crospovidone was added to above mass and mixed further to get a homogeneous blend. All other ingredients were added to above mass and granulated to obtain granules of valsartan. These granules were then blended with other excipients except lubricant. The granules were then lubricated using magnesium stearate and compressed to form active layer blend. Povidone was dissolved in IPA: water mixture with overhead stirring. A part of polyethylene oxide, a part of hydroxyl propyl methyl cellulose, hydroxyethyl cellulose, a part of crospovidone, microcrystalline cellulose were passed through the sieve and dry mixed in rapid mixer. The binder solution was added to the dry mix and the mass was granulated and subsequently dried in a fluidized bed dryer to get desired loss on drying. Sized dried granules were blended with all other excipients including lactose, microcrystalline cellulose, sodium bicarbonate and citric acid. The granules were then lubricated using magnesium stearate to form gastroretentive layer blend. A core bilayer gastroretentive tablet of valsartan was prepared by compressing the active layer and the gastroretentive layer.

The core tablets of valsartan were further coated by aqueous coating system comprising polyvinyl alcohol-based Opadry 200 Blue 200F105000 to achieve weight gain level of 4% and then imprinted.

### Example 2

This example summarizes a study that demonstrated that valsartan extended release formulation(160mg) as prepared in Example 1 has better pharmacokinetic profile as compared with valsartan immediate release (Diovan) 160 mg dose formulation.

The subjects selected were 44 healthy individuals, male and female, between the ages of 18 to 70 years inclusive, without a history of drug or alcohol abuse, and non-pregnant using adequate contraception.

The study conducted was a randomized, open label, crossover phase I study. Following initial screening, all the subjects entered a single blind placebo washout period. Both valsartan ER (160 mg) and Diovan® (160 mg) were administered as oral tablets. Blood samples were drawn pre-dose and then at 0.5, 0.75, 1, 1.5, 2, 2.5, 3, 4, 6, 8, 10, 12, 14, 16 and 24 hours post dose. Following the 24 hour time point, subjects were discharged from the PK unit and instructed to return to the PK for next product administration. A single blind placebo washout period of 7 days to replace treatment was used before switching to the next product as patients were evaluated for both the products. Study procedures for both the products were identical. After the final blood sample collection, end of study procedures were performed and subjects were discharged from the study unit. Pharmacokinetic analysis was determined by examining the amount of drug in the bloodstream over the course of 24 hours. Physical examinations, electrocardiograms, hematology/chemistry/urinalysis and recording of other vital signs were performed at screening, at every visit to PK unit and at the end of study. The log-transformed AUC₀₋₂₄ and Cₘₐₓ were analyzed using a mixed effect linear model.

The pharmacokinetics results as ratio of plasma concentrations of valsartan provided by the extended release formulation of example 1 to that provided by valsartan immediate release formulation (Diovan) are depicted in Table 2 below:

TABLE 2
Ratio of plasma concentrations of Valsartan ER and Diovan

| | Ratio of plasma concentration of |
|---|---|
| Time | formulation of example 1 to |
| (Hours) | Diovan |
| 0 | 0 |
| 0.5 | 0.037 |
| 0.75 | 0.053 |
| 1 | 0.066 |
| 1.5 | 0.10 |
| 2 | 0.17 |
| 2.5 | 0.26 |
| 3 | 0.47 |
| 4 | 0.84 |
| 6 | 1.31 |
| 8 | 1.58 |
| 10 | 2.31 |
| 12 | 2.52 |
| 14 | 2.24 |
| 16 | 2.41 |
| 24 | 2.25 |

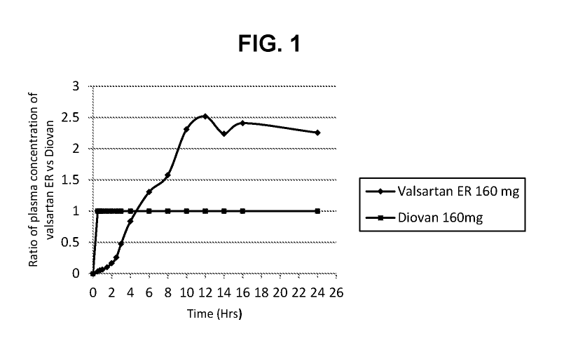

The above results are also depicted graphically in FIG. 1.

The results indicate that the ratio of plasma concentrations seen for valsartan extended release formulation of example 1 to that for immediate release formulation are greater than 1 over the time period of 8 hours to 24 hours after administration. In the time period of 16 hours to 24 hours the ratio is observed to be greater than 1.5. This shows that treatment of a cardiovascular condition by administering valsartan extended release formulation discussed in example 1 provides effective levels of plasma concentrations of valsartan than the immediate release formulation and thereby offers better extended protection in the high risk time period of 20 hours to 24 hours after administration.

1. A method of treating cardiovascular conditions comprising orally administering once a day to a human subject in need thereof the compound valsartan in a gastroretentive extended release dosage form,
wherein the gastroretentive extended release dosage form comprises:
a gastroretentive core comprising
(a) a therapeutically effective amount of about 160 mg;
(b1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg;
(f) a pharmaceutically acceptable excipient; and
at least one coating system surrounding the gastroretentive core; and
wherein the gastroretentive extended release dosage form provides at least twice the plasma concentration of valsartan compared to the plasma concentration of valsartan provided by an immediate release formulation of valsartan over a time period of 10 to 24 hours after administration in a single dose human pharmacokinetic study.

2. The method of claim 1 wherein the cardiovascular condition is selected from high blood pressure, heart failure, or heart attack.

3. The method of claim 1, wherein the gastroretentive extended release dosage faun provides at least twice the plasma concentration of valsartan compared to the plasma concentration provided by an immediate release formulation over a time period of 12 to 24 hours after administration in a single dose human pharmacokinetic study.

4. The method of claim 1 wherein the gastroretentive extended release dosage form provides at least twice the plasma concentration of valsartan compared to the plasma concentration provided by an immediate release formulation over a time period of 16 to 24 hours after administration in a single dose human pharmacokinetic study.

5. The method of claim 1 wherein the gastroretentive extended release dosage form provides at least twice the plasma concentration of valsasrtan compared to the plasma concentration provided by an immediate release formulation over a time period of 20 to 24 hours after administration in a single dose human pharmacokinetic study.

6. (canceled)

7. (canceled)

8. (canceled)

9. (canceled)

10. The method of claim 1 wherein the gastroretentive extended release dosage form comprises valsartan in a solubilized form.

11. A method of treating cardiovascular conditions comprising orally administering once a day to a human subject in need thereof the compound valsartan in a gastroretentive extended release dosage form,
wherein the gastroretentive extended release dosage form comprises:
a gastroretentive core comprising
(a) a therapeutically effective amount of valsartan of about 160 mg;
(b1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg;
(f) a pharmaceutically acceptable excipient; and
at least one coating system surrounding the gastroretentive core; and
wherein the ratio of mean plasma concentration of valsartan provided by the gastroretentive extended release dosage form to the mean plasma concentration of valsartan provided by an immediate release dosage form of valsartan over the 10-24 hour period after administration is greater than 2 in a single dose human pharmacokinetic study.

12. The method of claim 11 wherein the cardiovascular condition is selected from high blood pressure, heart failure, or heart attack.

13. The method of claim 11 wherein the ratio of mean plasma concentration of valsartan provided by the gastroretentive extended release dosage form to the mean plasma concentration of valsartan provided by an immediate release dosage form over the 12-24 hour period after administration is greater than 2 in a single dose human pharmacokinetic study.

14. The method of claim 11 wherein the ratio of mean plasma concentration of valsartan provided by the gastroretentive extended release dosage form to the mean plasma concentration of valsartan provided by an immediate release dosage form over the 16-24 hour period after administration is greater than 2 in a single dose human pharmacokinetic study.

15. The method of claim 11 wherein the ratio of mean plasma concentration of valsartan provided by the gastroretentive extended release dosage form to the mean plasma concentration of valsartan provided by an immediate release dosage form over the 20-24 hour period after administration is greater than 2 in a single dose human pharmacokinetic study.

16. (canceled)

17. (canceled)

18. (canceled)

19. The method of claim 11 wherein the gastroretentive extended release dosage form comprises valsartan in solubilized form.

20. A method of treating cardiovascular conditions comprising orally administering once a day to a human subject in need thereof the compound valsartan in a gastroretentive extended release dosage form,
wherein the gastroretentive extended release dosage form comprises:
a gastroretentive core comprising
(a) a therapeutically effective amount of valsartan of about 160 mg;
(b 1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg;
(f) a pharmaceutically acceptable excipient; and
at least one coating system surrounding the gastroretentive core; and
wherein the gastroretentive extended release dosage form provides at least twice as much area under the curve in the 16-24 hour time period compared to those the area under the curve provided by immediate release formulation of valsartan over the period of 16-24 hours after administration in a single dose pharmacokinetic study.

21. The method of claim 20 wherein the cardiovascular condition is selected from high blood pressure, heart failure, or heart attack.

22. A method of treating cardiovascular conditions comprising orally administering once a day to a human subject in need thereof valsartan in a gastroretentive extended release dosage form
wherein the gastroretentive extended release dosage form comprises:
a gastroretentive core comprising
(a) a therapeutically effective amount of valsartan of about 160 mg;
(b1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg;
(f) a pharmaceutically acceptable excipient; and
at least one coating system surrounding the gastroretentive core; and
wherein the gastroretentive extended release dosage form provides at least 20% of the total area under the curve (AUC) in the 16-24 hour time period after administration in a single dose pharmacokinetic study.

23. The method of claim 22 wherein the cardiovascular condition is selected from high blood pressure, heart failure, or heart attack.
